(19) United States
(12) Patent Application Publication
(10) Pub. No.: US 2012/0183531 A1
(43) Pub. Date: Jul. 19, 2012

(54) Methods for Inhibiting Yellow Color Formation in a Composition

(75) Inventors: Karin Lucas, Encinitas, CA (US); Kevin Maloney, Nashua, NH (US)

(73) Assignee: BIOGEN IDEE MA INC, CAMBRIDGE, MA (US)

(21) Appl. No.: 13/384,178

(22) PCT Filed: Jul. 13, 2010
(86) PCT No.: PCT/US10/41842

§ 371 Date: Apr. 2, 2012

Related U.S. Application Data
(60) Provisional application No. 61/225,455, filed on Jul. 14, 2009.

(51) Int. Cl. A61K 39/395; A61K 47/26; C07D 233/64; A61K 47/22; A61K 38/02; G01N 21/75

(52) U.S. Cl. 424/130.1; 514/1.1; 436/86; 436/98; 548/339.1; 514/784; 514/777

(57) ABSTRACT

The present invention is related to methods for preventing or retarding (i.e., inhibiting) yellow color or peroxide formation in a composition. The present invention is also related to methods of reducing or decreasing the amount of yellow color or peroxide in a composition. More specifically, the present invention relates to the use of an antioxidant, an oxygen scavenger, pH, a chelating agent, and/or at least two factors in the methods of the invention. The present invention is also related to methods for predicting the rate of yellow color or peroxide formation in a composition.

### BACKGROUND OF THE INVENTION

1. Field of the Invention

The present invention relates generally to the field of pharmaceutical protein formulations. Specifically, the present invention is related to methods for preventing or retarding (i.e., inhibiting) yellow color formation in a composition. The present invention is also related to methods of reducing or decreasing the amount of yellow color in a composition. The present invention also relates to predicting the rate of yellow color formation in a composition. In one embodiment, methods of the invention comprise use of an antioxidant, an oxygen scavenger, pH, and/or a chelating agent to inhibit or reduce yellow color formation. In another embodiment, methods of the invention comprise use of two or more factors to inhibit or reduce yellow color formation. In another embodiment, the present invention provides methods for predicting the rate of yellow color formation in a composition based on the presence of two or more factors in the composition.

2. Background Art

Histidine, citrate, phosphate, succinate, acetate, and Tris are commonly used to buffer pharmaceutical protein formulations. Histidine has excellent buffer capacity in the pH range typically used for biopharmaceuticals, pH 5.5-7.4, and has been found to stabilize some proteins against degradation. A drawback to the use of histidine as a buffer in liquid formulations is its propensity to change color from clear to yellow during storage. Despite the formation of a yellow color, a number of pharmaceutical protein formulations use histidine as a buffer, including, but not limited to, NORDITROPIN®, XOLAIR®, KEPIVANCE®, RECOMBINATE™, KOGENATE®, SYNAGIS®, RAPTIVA®, and HERCEPTIN®. Of these pharmaceutical protein formulations, the prescribing information for RECOMBINATE™, KOGENATE®, RAPTIVA®, and HERCEPTIN® mentions that the lyophilized or reconstituted protein formulations can be pale yellow. Furthermore, the histidine monograph from the European Pharmacopoeia indicates that histidine needs to meet certain color standards but does not need to be colorless.

The number of FDA-approved, commercial protein formulations containing histidine buffer mention having at least some yellow coloration might be thought to imply that formation of a yellow color is not detrimental to, or indicative of loss in, the efficacy of these biopharmaceuticals. In some industries, however, yellow color formation is associated with scorching, soiling, and general product degradation, e.g., textile, paint, and plastics industries. Further, in the chemical arts, compounds that are otherwise clear or white when pure, can have yellow coloration when less than pure; such as, for example, rapamycin. See e.g., U.S. Pat. No. 7,384,953.

At least one group has postulated that an accelerated loss in potency of a protein formulation containing histidine buffer was attributable to oxidation. See Subramanian, M., et al., AAPS Pharm Sci. 2001; 3(S1) 1884, AAPS Denver Poster Presentation. Subramanian, M., et al. analyzed the accelerated loss of potency of a humanized IgG2 monoclonal antibody formulated in histidine buffer plus TWEEN® 80 and found that the loss of potency was attributable to oxidation of both the histidine buffer and the monoclonal antibody by peroxides. Id. Further, Subramanian, et al. found that histidine oxidation products, including free radicals and 4(5)-imidazolecarboxaldehyde (4(5)-ICA), contributed to an accelerated loss of potency. Id. Subramanian, M., et al. investigated the effect of O₂, N₂, and EDTA on the formation of the histidine oxidation product 4(5)-ICA and found that N₂ prevented formation of 4(5)-ICA, while O₂ accelerated formation and EDTA had no effect on formation of 4(5)-ICA. Id. Subramanian, M., et al. did not however disclose any correlation between the oxidation of histidine buffer and the formation of a yellow color, nor did they disclose methods for preventing or reducing yellow color formation of a buffer that yellows during storage, such as histidine. Thus, there remains a need for methods for preventing, retarding, or reducing yellow color formation in buffers, such as histidine buffers, used in protein formulations (even if such methods are practiced only for aesthetic value).

### BRIEF SUMMARY OF THE INVENTION

The present invention is directed to new and useful methods for preventing or reducing yellow color formation in a composition. The methods include the use of an antioxidant, oxygen scavenger, or at least two factors to prevent or reduce yellow color formation. The present invention is also directed to new and useful methods for predicting the rate of yellow color formation in a composition.

In one embodiment, the present invention provides a method for preventing or retarding (i.e., inhibiting) yellow color formation in a composition, wherein the method comprises use of an antioxidant, oxygen scavenger, and/or chelating agent. In another embodiment, the present invention provides a method of reducing or decreasing the amount of yellow color in a composition, wherein the method comprises the use of an antioxidant, oxygen scavenger, and/or chelating agent. In particular embodiments, compositions to which methods of the invention are applied comprise solutions or formulations such as a buffer solution, a protein formulation, a solution containing a protein, and a solution containing an antibody.

In particular embodiments, the compositions comprise compounds such as histidine, citrate, phosphate, succinate, Tris, acetate, or any combination of two or more of these. In a preferred embodiment, the composition comprises histidine. In another embodiment, the composition further comprises an excipient, such as a polysorbate compound, polysorbate 20, polysorbate 80, NaCl, sucrose, glycerol, arginine, glycine, trehalose, mannitol, xylitol, lactose, sorbitol, a poloxamer, a glycol, CaCl₂, imidazole, benzyl alcohol, urea, leucine, isoleucine, threonine, glutamate or glutamic acid, phenylalanine, cresol, or any combination of two or more of these.

In some embodiments, the antioxidant or oxygen scavenger used in the methods of the invention is a compound such as methionine, ascorbic acid, glutathione, Vitamin A, Vitamin E, selenium, retinyl palmitate, cysteine, sodium sulfite, thioglycerol, thioglycolic acid, metabisulfite, or any combination of two or more of these. In a preferred embodiment, the antioxidant or oxygen scavenger is methionine. In some embodiments, the concentration of the antioxidant or oxygen scavenger is in a range of about 0.0001 mM to about 10000 mM, about 0.001 mM to about 1000 mM, about 0.01 mM to about 100 mM, and about 0.1 mM to about 10 mM.

Embodiments of the invention also comprise use of a chelating agent to inhibit or reduce yellow color formation in a composition. Chelating agents are known in the art and are commonly used, e.g., to remove trace metals from solutions. Exemplary chelating agents include, but are not limited to, EDTA (ethylenediaminetetraacetic acid); EGTA (ethyleneglycoltetraacetic acid); ascorbic acid, iminodiacetate; tetrasodium iminodisuccinate; citric acid; dicarboxymethylglutamic acid; EDDS (ethylenediaminedisuccinic acid); DTPMP.Na (hepta sodium salt of diethylene triamine penta or methylene phosphonic acid); malic acid; NTA (nitrilotriacetic acid); nonpolar amino acids (including, but not limited to, methionine); oxalic acid; phosphoric acid; polar amino acids (including, but not limited to, arginine, asparagine, aspartic acid, glutamic acid, glutamine, lysine, and ornithine); siderophores (including, but not limited to, Desferrioxamine B); and succinic acid. Chelating agents can also include, but are not limited to, chelators that are used for solution processing such as hydrolysed wool or a chelating resin, e.g., CHELEX® 20 or CHELEX® 100 resins (e.g. Bio-Rad Laboratories Hercules, Calif., USA).

In another embodiment, methods of the invention comprise decreasing exposure of the composition to oxygen. In some embodiments, the decrease in exposure of the composition to oxygen is performed by a means such as reducing the headspace gas content between the surface of the composition and a container closure; reducing ambient oxygen content; overlaying the composition with nitrogen; sparging the composition with nitrogen; or any combination of these. In another embodiment, the decrease in exposure of the composition to oxygen is performed by replacing headspace gas with a gas other than oxygen. In a preferred embodiment, the headspace gas is replaced with one or more inert gases (for example, but not limited to, nitrogen, helium, neon, argon, krypton, and xenon).

In one embodiment, methods of the invention comprise adjusting the pH of the composition. In some embodiments, the adjusted pH is in a range of about 7.5 to about 7.0; about 7.0 to about 6.5; about 6.0 or less; about 5.5 or less; and about 5.0 or less.

In another embodiment, methods of the invention comprise use of container coloration or packaging to protect compositions from exposure to light. In one embodiment, methods of the invention comprise use of a container coloration and packaging to protect compositions from exposure to light.

The present invention provides a method for preventing or retarding (i.e., inhibiting) yellow color formation in a composition, wherein the method comprises use of at least two factors to inhibit yellow color formation. The present invention also provides a method for reducing or decreasing the amount of yellow color in a composition, wherein the method comprises the use of at least two factors to reduce or decrease yellow color formation. In one embodiment, methods of the invention are applied to a composition comprising a solution or formulation such as a buffer solution, a protein formulation, a solution containing a protein, and a solution containing an antibody.

In some embodiments, the use of at least two factors comprises the use of any combination of two or more factors such as use of methionine, decreased oxygen exposure, NaCl, a polysorbate compound, polysorbate 20, polysorbate 80, arginine, a pH of about 5.0, a pH of about 5.5, a pH of about 6.0, a pH of about 6.5, and a pH of about 7.0. In particular embodiments, the use of at least two factors comprises the use of one or more combinations such as the use of methionine and decreased oxygen exposure; methionine and a pH of about 5; methionine and a pH of about 5.5; methionine and a pH of about 6; NaCl and polysorbate 80; and arginine and a pH of about 7.

In one embodiment, the reduction or decrease in the amount of yellow color in a composition is measured as a percent decrease in b* value. In some embodiments, the percent decrease in b* value is about 10%, about 20%, about 30%, about 40%, about 50%, about 60%, about 70%, about 80%, about 85%, about 90%, about 95% or about 99%. (See description further below for explanation of b* value).

The present invention provides a method for predicting or determining the rate of yellow color formation in a composition, wherein the predicting or deteiinining comprises the steps of incubating the composition at a specified range of temperatures, quantitating the amount of yellow color formed as a function of time and temperature, and extrapolating a prediction or determination of the rate of yellow color formation in said composition for any temperature inside or outside the specified range of temperatures. In one embodiment, the composition of this method comprises a solution or formulation such as a buffer solution, a protein formulation, a solution containing a protein, and a solution containing an antibody.

In one embodiment, the method for predicting or determining the rate of yellow color formation further comprises the steps of preparing a range of concentrations of the composition and extrapolating a prediction or determination of the rate of yellow color formation in the composition for any concentration inside or outside the specified range of concentrations.

In some embodiments, the composition comprises a compound such as histidine, citrate, phosphate, succinate, Tris, acetate, or any combination of two or more of these. In one embodiment, the rate of yellow color formation in the composition is predicted or determined as a function of solution storage temperature. In a preferred embodiment, the rate of yellow color formation in the composition is predicted or determined as a function of solution storage temperature for any temperature, in 1° C. increments, between about −80° C. [minus 80° C.] and about +100° C. In another embodiment, the prediction or determination is calculated or based on a two-factor interaction. In one embodiment, the prediction or determination is based on Arrhenius modeling.

### BRIEF DESCRIPTION OF THE DRAWINGS/FIGURES

FIG. 1A shows a graph of the b* values for the Y (squares) and BY (diamonds) EP color standards as measured by the HunterLab ColorQuest XE spectrophotometer versus the fold-dilutions of the color standards, with a visual representation of each set of color standard fold-dilutions inset.

FIG. 1B shows a graph of the b* values as measured by the HunterLab ColorQuest XE spectrophotometer versus the closest fold-dilution of the Y EP color standard.

FIG. 2A shows a graph of the percentage of intact antibody versus the change in b* value. Clear triangles correspond to histidine buffer comprising an antibody. Clear circles correspond to histidine buffer comprising an antibody and polysorbate 80. Filled circles correspond to histidine buffer comprising an antibody and methionine. Filled triangles correspond to histidine buffer comprising an antibody, polysorbate 80, and methionine.

FIG. 2B shows a graph of the percentage of high molecular weight aggregates of antibody versus the change in b* value. Clear triangles correspond to histidine buffer comprising an antibody. Clear circles correspond to histidine buffer comprising an antibody and polysorbate 80. Filled circles correspond to histidine buffer comprising an antibody and methionine. Filled triangles correspond to histidine buffer comprising an antibody, polysorbate 80, and methionine.

FIG. 2C shows a graph of the percentage of oxidized antibody versus the change in b* value. Clear triangles correspond to histidine buffer comprising an antibody. Clear circles correspond to histidine buffer comprising an antibody and polysorbate 80. Filled circles correspond to histidine buffer comprising an antibody and methionine. Filled triangles correspond to histidine buffer comprising an antibody, polysorbate 80, and methionine.

FIG. 3A shows a graph of the Arrhenius analysis of 200 mM histidine, pH 7.0, by plotting the natural log of k (the Boltzmann constant) versus 1/T (the absolute temperature) to determine the activation energy (Ea) of histidine at 200 mM.

FIG. 3B shows a graph of the order of reaction of histidine at 25° C., which is determined by plotting the natural log of the yellowing rate versus the natural log of the concentration of histidine.

FIG. 3C shows a graph of the order of reaction of histidine at 55° C., which is determined by plotting the natural log of the yellowing rate versus the natural log of the concentration of histidine.

FIG. 4 shows a graph of the change in b* value versus the time in days that a 200 mM histidine buffer was incubated at 40° C. in the presence or absence of polysorbate 80, at either pH 5.0 or 7.0. Squares correspond to histidine buffer with polysorbate 80 at pH 7.0. Diamonds correspond to histidine buffer at pH 7.0. Circles correspond to histidine buffer with polysorbate 80 at pH 5.0. Triangles correspond to histidine buffer at pH 5.0.

FIG. 5 shows a graph of the change in b* value versus the time in days that a 200 mM histidine buffer was incubated at 40° C. in the presence of various gases in the headspace volume. Crosses (X′s) correspond to an overlay of 60% O₂ in the headspace volume. Clear squares correspond to an overlay of 100% O₂ in the headspace volume. Filled triangles correspond to an ambient gas content in the headspace volume. Filled squares correspond to an overlay of N₂ in the headspace volume. Filled diamonds correspond to sparging of the histidine buffer with N₂.

FIG. 6 shows a graph of the change in peroxide concentration versus time in days when various 200 mM histidine-containing solutions (with or without a 1% polysorbate compound and/or methionine) were stored at pH 6.5 with various volumes of headspace containing either air or nitrogen (N₂) gas.

FIG. 7 shows a graph of the change in b* value versus time in days when various 200 mM histidine-containing solutions stored with various volumes of headspace containing air or nitrogen (N₂) gas were measure in vials.

FIG. 8 shows a graph of the change in b* value versus time in days when various 200 mM histidine-containing solutions stored with various volumes of headspace containing air or nitrogen (N₂) gas were measure in cuvettes.

FIG. 9 shows a graph of the change in b* value versus time in days when solutions of 200 mM histidine, 100 mM glycine, 100 mM glycerol, 0.05% Tween 80, at pH 7 with varying amounts of EDTA were incubated at 60° C. Solution color was measured using the Hunter LAB ColorQuest XE instrument.

FIG. 10 shows a graph of the change in b* value versus time in days when solutions of 200 mM histidine, 100 mM glycine, 100 mM glycerol, 0.05% Tween 80, at pH 7 with varying amounts of methionine were incubated at 60° C. Solution color was measured using the Hunter LAB ColorQuest XE instrument.

### DETAILED DESCRIPTION OF THE INVENTION

### Definitions

Terms are used herein as generally used in the art, unless otherwise defined as follows. In the case where there are two or more definitions of a term which is used and/or accepted within the art, the definition of the term as used herein is intended to include all such meanings unless explicitly stated to the contrary. All publications, patents and other references mentioned herein are incorporated by reference in their entireties for all purposes.

The term “yellow color” refers to one of the primary colors in the visible spectrum. For example, a yellow colored substance can absorb light in the range of approximately 420-430 nm. Yellow color can be evaluated subjectively, e.g., visually, or objectively, e.g., using a spectrophotometer or a colorimeter. A number of standards and formulas have been developed to evaluate color both subjectively and objectively and can be used to measure yellow color. An example of color scales that can be used to measure yellow color include, but are not limited to, the CIE (International Commission on Illumination) L*a*b* color scale, the CIE L*c*h* color scale, and the Hunter L, a, b color scale. These color scales are based on the Opponent-Colors Theory, which assumes that receptors in the human eye perceive color as a pair of opposites: light-dark (L* value), red-green (a* value), and yellow-blue (b* value) (see “Hunter L, a, b Versus CIE 1976 L*a*b*,” Application Notes, Insight on Color Vol. 13, No. 2 (2008)). Thus, as used herein, the term “b* value” refers to the yellowness or blueness of the composition. A positive b* (+b*) value refers to the yellowness of the composition, whereas a negative b* (−b*) value refers to the blueness of the composition. Additional information regarding color scales and color measurement can be obtained from, e.g., the CIE (www.cie.co.at) or HunterLab (www.hunterlab.com) (Hunter Associates Laboratory, Inc., Reston, Va., USA).

As used herein, the term “antioxidant” refers to any compound or substance that inhibits or slows oxidation or reactions promoted by oxygen and peroxides.

As used herein, the term “oxygen scavenger” refers to any compound or substance that consumes or renders inactive the oxygen impurities in a composition.

The term “buffer” refers to a compound that resists changes in pH by the action of its acid-base conjugate components.

The term “formulation” includes any solutions, suspensions, or dosage forms in which different substances are combined. As used in the context of the invention, one formulation includes, for example, a protein formulation.

The term “solution” refers to a mixture of one or more liquids with a gas, a solid, or both a gas and a solid. As used in the context of the invention, a solution includes a buffer solution and a solution can contain a protein or a solution can contain an antibody.

As used herein, the term “protein” encompasses “peptides,” “dipeptides,” “tripeptides,” “oligopeptides,” “polypeptides,” “amino acid chain,” or any other term used to refer to a chain or chains of two or more amino acids, and the term “protein” may be used instead of, or interchangeably with any of these terms. Thus, protein is intended to encompass a singular “polypeptide” as well as plural “polypeptides,” and refers to a molecule composed of monomers (amino acids) linearly linked by amide bonds (also known as peptide bonds). Because the term protein refers to any chain or chains of two or more amino acids, protein does not refer to a specific length of the product. Thus, the term protein is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A protein may be derived from a natural biological source or produced by recombinant technology, but is not necessarily translated from a designated nucleic acid sequence. It may be generated in any manner, including by chemical synthesis. Because an antibody encompasses a chain of two or more amino acids, the term protein includes an antibody.

As used herein, “antibody” means an intact immunoglobulin, or an antigen-binding fragment thereof Antibodies or antigen-binding fragments, variants, or derivatives thereof (which may be part of compositions, solutions, or formulations to which methods of the invention are applied) include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domains, fragments produced by Fab expression libraries, and anti-idiotypic (anti-Id) antibodies. Immunoglobulin or antibody molecules can be of any type (without limitation, e.g., IgG, IgE, IgM, IgD, IgA, and IgY), any class (without limitation, e.g., IgG1, IgG2, IgG3, IgG4, IgA1 and IgA2) or any subclass of immunoglobulin molecule. Multispecific antibodies and antigen-binding fragments (e.g., bispecific, trispecific, or of greater multispecificity) include those antibodies and antigen-binding fragments that recognize and bind to two or more different epitopes present on one or more different antigens (e.g., proteins) at the same time. Thus, whether an antibody is “monospecific” or “multispecific,” e.g., “bispecific,” refers to the number of different epitopes (i.e., “bi”=2, “multi”=more than 1) with which a binding polypeptide reacts.

As used herein, “excipient” is intended to mean anything other than an active ingredient, e.g., a protein or an antibody, in a composition. Thus, for example, excipient refers to any more or less inert substance added to a composition in order to confer a suitable consistency, form, or stability to the composition. An excipient also can be used as a vehicle or carrier for an active ingredient. Types of excipients include, but are not limited to, antiadherents, binders, coatings, disintegrants, fillers, diluents, flavors, colors, glidants, lubricants, preservatives, sorbents, compression aids, suspending agents, dispersing agents, surfactants, and sweeteners.

It is to be noted that the terms “a” or “an” refer to both singular and plural forms of the terms; for example, “an antioxidant,” is understood to represent one or more antioxidants. As such, the terms “a” (or “an”), “one or more,” and “at least one” can be used interchangeably herein.

As used herein, the term “about” allows for the degree of variation inherent in the methods and in the instrumentation used for measurement or quantitation. For example, depending on the level of precision of the instrumentation used, standard error based on the number of samples measured, and rounding error, the term “about” includes, without limitation, ±10%.

### Description of the Methods

A number of pharmaceutical protein formulations have been described as having a yellow color and include, but are not limited to, RECOMBINATE™, KOGENATE®, RAPTIVA®, and HERCEPTIN®. Each of these formulations contains in common at least histidine buffer, which according to the European Pharmacopoeia can form a yellow color. Other formulations have been shown to form a yellow color, including, e.g., citrate formulations when heated to high temperatures. The present invention provides new and useful methods for preventing, retarding, or reducing yellow color formation in such formulations. Thus, the methods of the invention include a method for preventing, retarding or reducing yellow color formation in a composition, wherein the method comprises use of an antioxidant, oxygen scavenger and/or chelating agent in said composition. The present invention also provides a method of reducing or decreasing the amount of yellow color in a composition, wherein the method comprises the use of an antioxidant, oxygen scavenger, or chelating agent in said composition.

Compositions included in the methods of the invention encompass, without limitation, a solution or formulation such as a buffer solution, a protein formulation, a solution containing a protein, and a solution containing an antibody. However, other compositions that form a yellow color, which can be prevented or reduced by the methods of the invention, are included within the scope of the invention. In certain embodiments, the compositions comprise a buffer. Many buffers are known in the art for use in buffer solutions, protein formulations, or solutions containing proteins or antibodies and include, but are not limited to, histidine, citrate, phosphate, succinate, tris(hydroxymethyl)aminomethane (Tris), acetate, glycine, aconitate, maleate, phthalate, cacodylate, barbitol, 2-(N-morpholino)ethanesulfonic acid (MES), bis(2-hydroxyethyl)imino-tris-(hydroxymethyl)methane (Bistris), N-(2-Acetamido)iminodiacetic acid (ADA), piperazine-N,N′-bis(2-ethanesulfonic acid) (PIPES), 1,3-bis[tris(hydroxymethyl)-methylamino]propane (Bistrispropane), N-(Acetamido)-2-aminoethanesulfonic acid (ACES), 3-(N-morpholino)propanesulfonic acid (MOPS), N,N-bis(2-hydroxyethyl)-2-amino-ethanesulfonic acid (BES), N-tris(hydroxymethyl)methyl-2-amino-ethanesulfonic acid (TES), N-2-hydroxyethylpiperazine-N-ethanesulfonic acid (HEPES), N-2-hydroxyethylpiperazine-N-propanesulfonic acid (HEPES), N-tris(hydroxymethyl)methylglycine (Tricine), N,N-bis(2-hydroxyethyl)glycine (Bicine), glycylglycine, N-tris(hydroxymethyl)methyl-3-amino-propanesulfonic acid (TAPS), 1,3-bis[tris(hydroxymethyl)-methylamino]propane (Bistrispropane). Suitable buffers for parenteral compositions include those compounds selected from the group consisting of histidine, citrate, phosphate, succinate, Tris, acetate, and any combination of two or more thereof. In a preferred embodiment, the composition comprises histidine.

Compositions included in the methods of the invention can further encompass an excipient. Many excipients are known in the art for use in buffer solutions, protein formulations, or solutions containing proteins or antibodies and include, but are not limited to, excipients selected from the following groups: antiadherents, binders, coatings, disintegrants, fillers, diluents, flavors, colors, glidants, lubricants, preservatives, sorbents, compression aids, suspending agents, dispersing agents, surfactants, and sweeteners. Non-limiting examples of such excipients include a polysorbate compound, polysorbate 20, polysorbate 80, NaCl, sucrose, glycerol, arginine, glycine, trehalose, mannitol, xylitol, lactose, sorbitol, a poloxamer, a glycol, CaCl₂, imidazole, benzyl alcohol, urea, leucine, isoleucine, threonine, glutamate or glutamic acid, phenylalanine, cresol, magnesium stearate, microcrystalline cellulose, starch (corn), silicon dioxide, titanium dioxide, stearic acid, sodium starch glycolate, gelatin, talc, calcium stearate, pregelatinized starch, hydroxypropyl methylcellulose, OPA products (coatings and inks), croscarmellose, hydroxypropyl cellulose, ethylcellulose, calcium phosphate (dibasic), crospovidone, and shellac (and glaze). As used herein, a polysorbate compound includes those compounds encompassed within the group of polyoxyethylene sorbitan fatty acid esters, which are a series of partial fatty acid esters of sorbitol and its anhydrides copolymerized with approximately 20, 5, or 4 moles of ethylene oxide for each mole of sorbitol and its anhydrides (Handbook of Pharmaceutical Excipients 580 (Rowe, R. C., et al. eds 5^{th} ed. 2006)). As used herein, a poloxamer includes a series of closely related block copolymers of ethylene oxide and propylene oxide (Handbook of Pharmaceutical Excipients 535 (Rowe, R. C., et al. eds 5^{th} ed. 2006)). As used herein, a glycol refers to any of a class of organic compounds belonging to the alcohol family and includes, but is not limited to, ethylene glycol, propylene glycol, hexylene glycol, polyethylene glycol, and polypropylene glycol.

In some embodiments, preferred excipients include compounds such as a polysorbate compound, polysorbate 20, polysorbate 80, NaCl, sucrose, glycerol, arginine, glycine, trehalose, mannitol, xylitol, lactose, sorbitol, a poloxamer, a glycol, CaCl₂, imidazole, benzyl alcohol, urea, leucine, isoleucine, threonine, glutamate or glutamic acid, phenylalanine, cresol, or any combination of two or more of these.

The methods of the invention comprise the use of an antioxidant, oxygen scavenger, and/or chelating agent to prevent or reduce the yellow color formation of a composition. A number of antioxidants or oxygen scavengers are known in the art and include, but are not limited to, Vitamin E, alpha tocopherol, ascorbyl palmitate, butylated hydroxyanisole, butylated hydroxytoluene, chelating agents, citric acid, erythorbic acid, ethyl oleate, fumaric acid, malic acid, monothioglycerol, phosphoric acid, propionic acid, propyl gallate, sodium ascorbate, sodium bisulfate, thymol, methionine, ascorbic acid, glutathione, Vitamin A, selenium, retinyl palmitate, cysteine, sodium sulfite, thioglycerol, thioglycolic acid, and metabisulfite. In one embodiment of the methods of the invention, the antioxidant or oxygen scavenger is selected from the group consisting of methionine, ascorbic acid, glutathione, Vitamin A, Vitamin E, selenium, retinyl palmitate, cysteine, sodium sulfite, thioglycerol, thioglycolic acid, metabisulfite, and any combination of two or more thereof. In a preferred embodiment, the antioxidant or oxygen scavenger is methionine.

The concentration of the antioxidant, oxygen scavenger, and/or chelating used in the methods of the present invention depends, in part, upon the particular antioxidant(s), oxygen scavenger(s), and/or chelating agent chosen, but can otherwise readily be determined by those of skill in the art. For example, the concentration of the antioxidant, oxygen scavenger, and/or chelating agent can range anywhere from femtomolar to molar quantities for the compositions within the scope of the methods of the present invention. In some embodiments, the concentration of antioxidant, oxygen scavenger, and/or chelating agent is in a range of about 0.0001 mM to about 10000 mM, about 0.001 mM to about 1000 mM, about 0.01 mM to about 100 mM, and about 0.1 mM to about 10 mM.

In addition to using antioxidants, oxygen scavengers, and/or chelating agents to prevent or reduce yellow color formation of a composition, the methods of the invention further comprise decreasing exposure of the composition to oxygen. Various means for decreasing exposure of compositions to oxygen are known in the art and range from means such as using air-tight containers to reducing or exchanging the gas in the headspace volume of the containers. Suitable means for decreasing exposure of a composition to oxygen include means such as reducing the headspace gas content between the surface of the composition and a container closure; reducing ambient oxygen content; overlaying the composition with nitrogen; sparging the composition with nitrogen; or any combination of one or more of these. In one embodiment, the decrease in exposure of the composition to oxygen is performed by replacing headspace gas with a gas other than oxygen. In another embodiment, the headspace is filled with an inert gas (for example, but not limited to, nitrogen, helium, neon, argon, krypton, and xenon).

Other means contemplated to prevent or reduce yellow color formation of a composition include adjusting the pH of the composition. To the extent that a protein formulation or a solution containing a protein or an antibody can tolerate a change in pH, a pH can be chosen that prevents or reduces yellow color formation while preserving the protein's or antibody's structure and function. In one embodiment, the pH is adjusted to a physiologic pH. In some embodiments, the adjusted pH is in a range of about 7.5 to about 7.0; about 7.0 to about 6.5; about 6.0 or less; about 5.5 or less; and about 5.0 or less.

Additional means contemplated to inhibit or reduce yellow color formation of a composition include reducing or limiting exposure of the solution to light. Suitable means for protecting a composition from light includes, but is not limited to, use of a container coloration and/or packaging. Non-limiting examples of such means for protecting compositions from light are known in the art, and include, for example, brown or other dark-colored glass or plastic containers and foil or similar enclosures.

Methods of the invention comprise use of a chelating agent to prevent or reduce yellow color formation of a composition. Chelating agents are known in the art and are commonly used, e.g., to remove trace metals from solutions. Exemplary chelating agents include, but are not limited to, EDTA (ethylenediaminetetraacetic acid); EGTA (ethyleneglycoltetraacetic acid); ascorbic acid, iminodiacetate; tetrasodium iminodisuccinate; citric acid; dicarboxymethylglutamic acid; EDDS (ethylenediaminedisuccinic acid); DTPMP.Na (hepta sodium salt of diethylene triamine penta or methylene phosphonic acid); malic acid; NTA (nitrilotriacetic acid); nonpolar amino acids (including, but not limited to, methionine); oxalic acid; phosphoric acid; polar amino acids (including, but not limited to, arginine, asparagine, aspartic acid, glutamic acid, glutamine, lysine, and ornithine); siderophores (including, but not limited to, Desferrioxamine B); and succinic acid. Chelating agents can also include, but are not limited to, chelators that are used for solution processing such as hydrolysed wool or a chelating resin, e.g., CHELEX® 20 or CHELEX® 100 resins.

The methods of the present invention also include a method for preventing or retarding yellow color formation in a composition, wherein the method comprises use of at least two factors to prevent or retard yellow color formation. The present invention also provides a method of reducing or decreasing the amount of yellow color in a composition, wherein the method comprises the use of at least two factors to reduce or decrease yellow color formation. It has surprisingly been found that, in some instances where use of one factor did not inhibit or reduce yellow color formation of a composition, the addition of a second factor did inhibit or reduce yellow color formation of a composition (see Table 2). Thus, in one embodiment, the use of at least two factors comprises the use of any combination of two or more factors such as the use of methionine, decreased oxygen exposure, NaCl, a polysorbate compound, polysorbate 20, polysorbate 80, arginine, a pH of about 5.0, a pH of about 5.5, a pH of about 6.0, a pH of about 6.5, and a pH of about 7.0. In another embodiment, the use of at least two factors comprises the use of one or more combinations of factors such as use of methionine and decreased oxygen exposure; methionine and a pH of about 5; methionine and a pH of about 5.5; methionine and a pH of about 6; NaCl and polysorbate 80; and arginine and a pH of about 7.

Methods of the present invention include methods wherein the reduction or decrease in the amount of yellow color in a composition is measured as a percent decrease in b* value. The measurement of the change in b* values can be performed using a colorimeter or spectrophotometer, using techniques known in the art. In one embodiment, the percent decrease in b* value is greater than 0%, in 1% increments up to 100%. In some embodiments, the percent decrease in b* value is about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and about 99%.

Methods of the present invention also include a method for predicting or deteimining the rate of yellow color formation in a composition, wherein the predicting or determining comprises the steps of incubating the composition at a specified range of temperatures, quantitating the amount of yellow color foamed as a function of time and temperature, and extrapolating a prediction or determination of the rate of yellow color formation in said composition for any temperature inside or outside the specified range of temperatures. In one embodiment, the method for predicting or determining the rate of yellow color formation further comprises the steps of preparing a range of concentrations of the composition and extrapolating a prediction or determination of the rate of yellow color formation in said composition for any concentration inside or outside the specified range of concentrations. Methods are known in the art for carrying out such steps.

In a further embodiment, the rate of yellow color formation in the composition is predicted or determined as a function of solution storage temperature. Solution storage temperatures can range over those temperatures in which the rate of yellow color formation can be decreased, to those temperatures in which the rate of yellow color formation can be increased. Such temperatures should not, however, interfere with the stability of the buffer solutions, protein formulations, and solutions containing a protein or an antibody. Thus, in a preferred embodiment, the rate of yellow color formation in the composition is predicted or determined as a function of solution storage temperature for any temperature, in 1° C. increments, between about −80° C. [minus 80° C.] and about +100° C.

In another embodiment, the prediction or determination is calculated or based on a two-factor interaction. In one embodiment, the prediction or determination is based on Arrhenius modeling.

### EXAMPLES

### Example 1

### A System for the Measurement of Yellow Color Formation Based on Yellow Color Standards

The HunterLab color system can be used to measure spectrophotometrically the degree of solution yellowing in a cuvette or in vials. EP color standards were created as described in section 2.2.2 of the European Pharmacopoeia “Degree of Coloration of Liquids”. The EP standard color stock solutions were purchased from Ricca Chemical Company. The Y and BY EP standards were measured using the HunterLab ColorQuest XE instrument both in a 1 cm path-length, quartz cuvette as well as in 10 mL glass vials. The b* color value was plotted against the fold-dilution used to create the color standards.

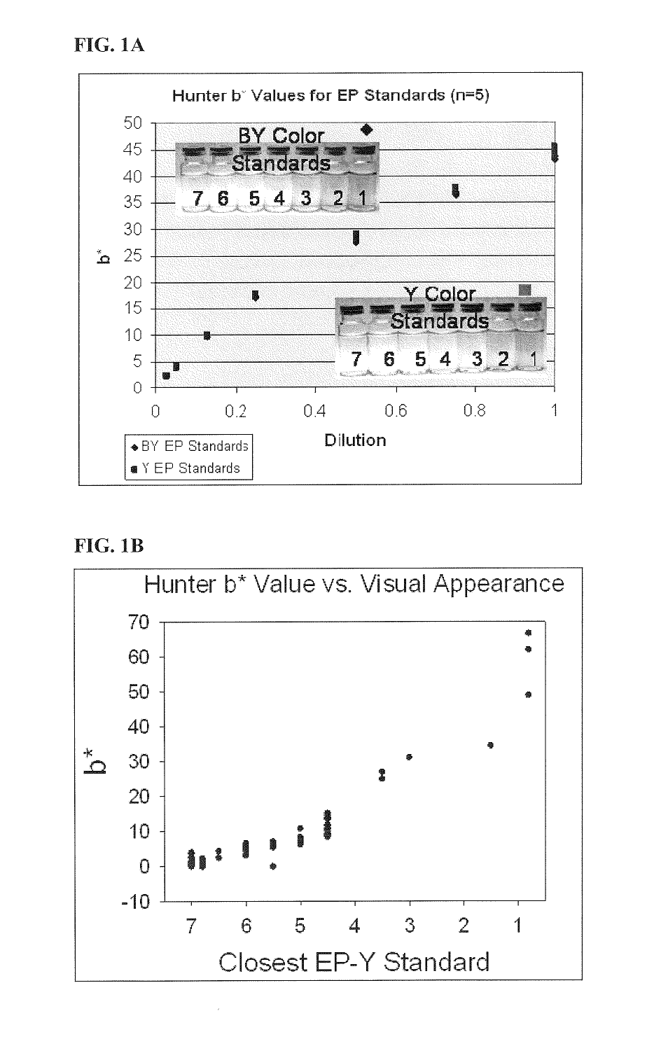

Analysis of the Y (squares) and BY (diamonds) EP color standards indicated a linear increase in b* values with increasingly yellow solutions up to b* values of 45 (FIG. 1A). Visual assessment of samples correlated well with b* values with the exception of very yellow samples where instrument linearity decreased (FIG. 1B). The HunterLab color measurement system facilitated a more quantitative and sensitive monitoring of color change over time than traditional visual appearance testing. Thus, such spectrophotometric systems can be used to determine the yellow color of a composition.

### Example 2

### Design of Experiments (DOE) Screen of Yellow Color Formation of Histidine Compositions Containing Common Excipients

In order to determine the effect of excipients and other formulation factors on the yellowing of any composition, screens can be designed by any number of methods, including statistical analysis programs. To test the effect of nine formulation factors on histidine yellowing, Design Expert 6.0.5 (Stat-Ease) was used to create a D-Optimal experimental design (see Table 1). The design included 55 formulations and had the statistical power to enable analysis of 2-factor interactions. Formulations were created using the following reagents: L-histidine (J. T. Baker), L-histidine monohydrochloride (J. T. Baker), sodium chloride (Sigma-Aldrich or Fisher Scientific), sucrose (high purity, low endotoxin, beet-derived from Ferro Pfanstiehl), L-methionine (Sigma), L-arginine hydrochloride (J. T. Baker), glycine (J. T. Baker), glycerol (J. T. Baker), polysorbate 80 (J. T. Baker (Phillipsburg, N.J., USA) and NOF America (White Plains, N.Y., USA)), polysorbate 20 (EMD Chemicals (Gibbstown, N.J., USA) and NOF America), and a typical monoclonal antibody produced by Biogen Idec.

TABLE 1
Reagents used in DOE parameter screen.

| Parameter | Level 1 | Level 2 | | Level 3 | Comments |
|---|---|---|---|---|---|
| Polysorbate | 0 | 0.05% | (w/v) | 0.05% | Source of peroxides |
| 80 | | | J. T. | NOF | |
| | | | Baker | | |
| pH | 5.0 | 7.0 | | | Buffer ion reactivity |
| NaCl | 0 | 150 | mM | | Common excipient |
| Sucrose | 0 | 5% | (w/v) | | Source of reducing |
| | | | | | impurities |
| Glycerol | 0 | 150 | mM | | Reactive oxygen |
| | | | | | species |
| Methionine | 0 | 10 | mM | | Antioxidant |
| Arginine | 0 | 150 | mM | | Common excipient |
| Glycine | 0 | 100 | mM | | Common excipient |
| Monoclonal | 0 | 25 | mg/mL | | Model protein |
| Antibody | | | | | |

10 mL of each formulation was filled into 10 mL glass vials, 20 mm opening, Schott, 8412-B glass cane. The vials were stoppered using grey butyl rubber, TEFLON®—2 coated, 20 mm stoppers from West Pharmaceutical Services. The vials were incubated at 2-8° C., 25° C./60% RH, or 40° C./75% RH for 18 months and the degree of yellowing was tested periodically by Hunter Lab analysis of the intact vials.

As shown in Table 2, the DOE parameter screen found that histidine buffer yellowing was accelerated by protein, polysorbate 80, glycerol, glycine, and a higher pH (pH 7.0 was more yellow than pH 5.0), when added individually to the histidine buffer, while methionine generally retarded yellowing. When looking at 2-factor interactions, the combination of NaCl and polysorbate 80 was found to decrease yellowing of the histidine buffer, as was the combination of arginine and a pH of 7.0.

TABLE 2
Results of DOE parameter screen for vials incubated at 25° C./60% RH.

| | | | 2-Factor | | |
|---|---|---|---|---|---|
| Factor | Effect on Yellowing | Prob > F | Interactions | Effect on Yellowing | Prob > F |
| Polysorbate 80 | Increases yellowing | <0.0001 | Glycine | Further increases | 0.0008 |
| | J. T. Baker = NOF | | | yellowing | |
| pH | pH 7.0 more yellow | <0.0001 | | | |
| | than pH 5.0 | | | | |
| NaCl | | | Glycine | Further increases | 0.0043 |
| | | | | yellowing | |
| | | | Polysorbate 80 | Decreases yellowing | <0.0001 |
| | | | Glycerol | Further increases | <0.0001 |
| | | | | yellowing | |
| Sucrose | | | | | |
| Glycerol | Increases yellowing | <0.0001 | | | |
| Methionine | Decreases yellowing | <0.0001 | | | |
| Arginine | | | pH | Decreases yellowing at | <0.0001 |
| | | | | pH 7.0 but not pH 5.0 | |
| Glycine | Increases yellowing | <0.0001 | | | |
| Monoclonal | Increases yellowing | <0.0001 | | | |
| Antibody | | | | | |

Prob > F indicates the probability that the result is due to noise.
Only P-values <0.05 were considered significant.

### Example 3

### Analysis of Product Quality in Relation to Yellow Color Formation

To determine whether yellow color formation of a histidine buffer affects the product quality of a typical monoclonal antibody (mAb) produced by Biogen Idec, samples of the mAb were incubated in histidine buffer comprising additional excipients. Other samples of the mAb were incubated in the same histidine buffer plus polysorbate 80, methionine, or polysorbate 80 and methionine. Intact antibody analysis was performed by LABCHIP® 90 gel chip analysis. High molecular weight aggregate analysis was performed by size exclusion chromatography using a TSK-GEL® G3000SW_{XL} column and guard column from Tosoh Bioscience (0.1 M sodium phosphate/0.2 M sodium chloride, pH 6.8 mobile phase, 0.5 mL/min flow rate, 60 minute separation with detection at 280 nm). Oxidized antibody was determined by LC-MS focused peptide map analysis.

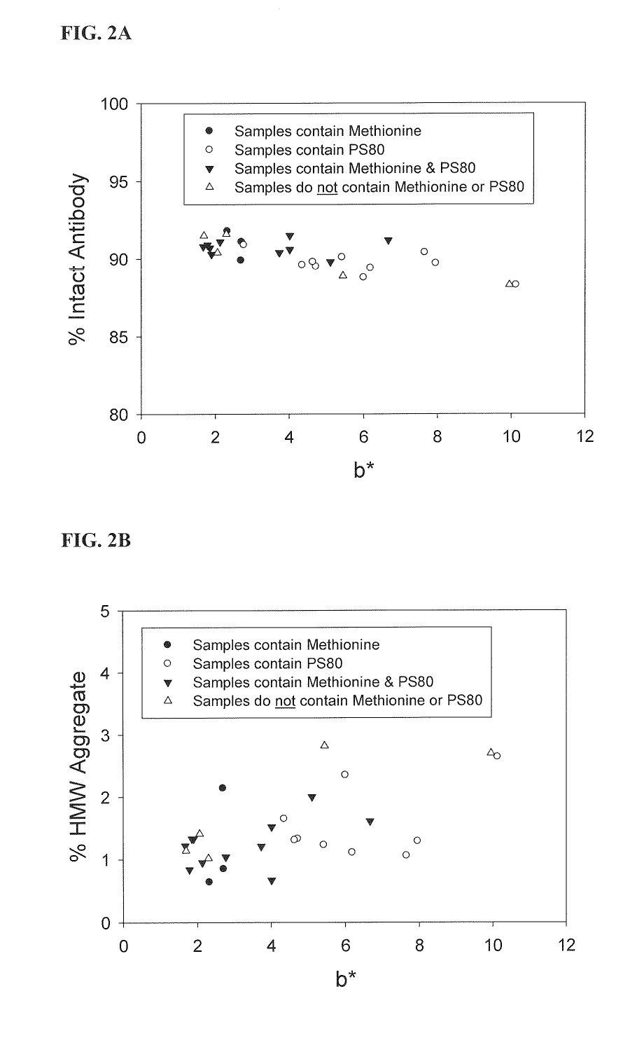

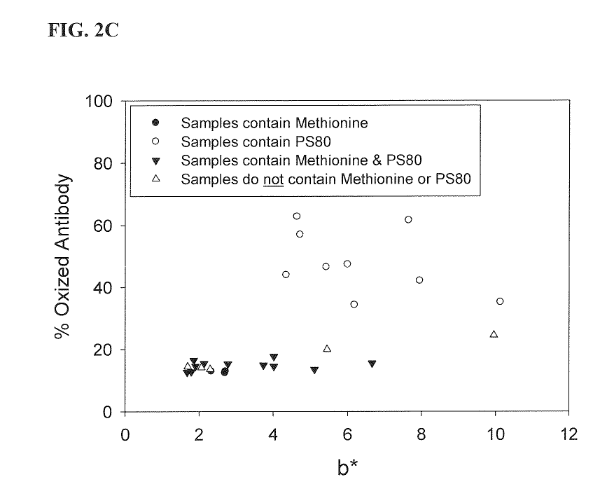

As shown in FIG. 2, significant changes in solution color do not necessarily correlate to changes in other product quality attributes. Although there were visually detectable changes in b* values, there was not a significant change in mAb fragmentation for samples of mAb incubated in histidine buffer (clear triangles), histidine buffer plus polysorbate 80 (clear circles), histidine buffer plus methionine (filled circles), or histidine buffer plus polysorbate 80 and methionine (filled triangles) (FIG. 2A). Formulations with similar changes in color had significantly different levels of aggregated (FIG. 2B) or oxidized mAb (FIG. 2C). For example, samples of the mAb incubated in histidine buffer plus polysorbate 80 (clear circles) showed higher levels of oxidized mAb (FIG. 2C). However, the weak correlation between solution yellowing and other product quality attributes suggests that product color is not necessarily predictive of product quality.

### Example 4

### Kinetic Evaluation of Yellow Color Formation in the Absence of Protein

To evaluate the kinetics of yellow color formation of histidine buffer, factors affecting histidine buffer yellowing, such as concentration and solution storage temperature, were analyzed in the absence of protein. Formulations for kinetic analysis containing either 20 mM, 100 mM, or 200 mM histidine were filled into vials and incubated at 2-8° C., 25° C./60% RH, 40° C./75% RH, or 55° C. Color values were periodically measured spectrophotometrically using a HunterLab ColorQuest XE instrument. Measurements were performed on the intact vials, and the vials were then returned to the incubation chambers.

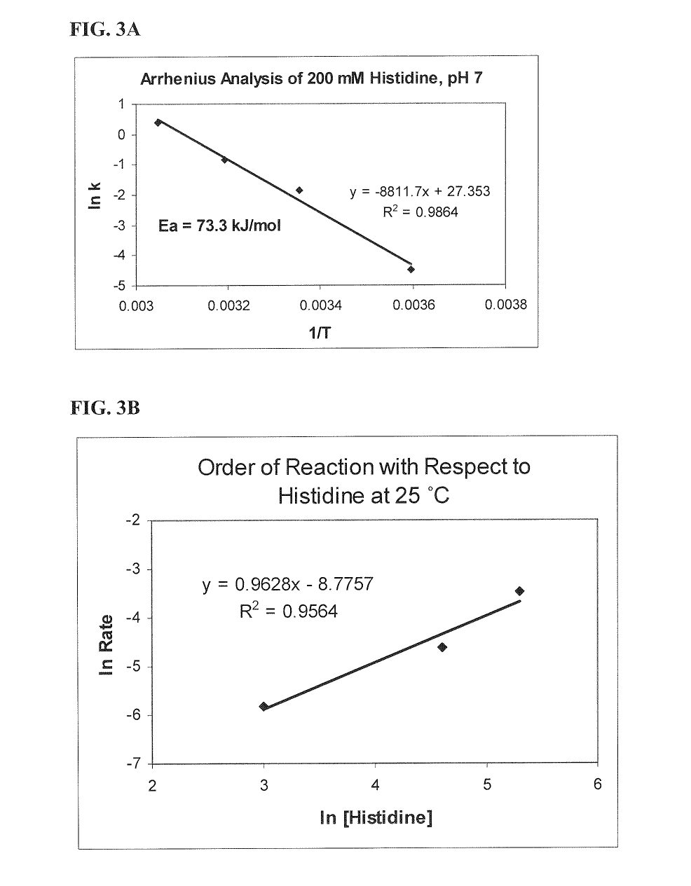

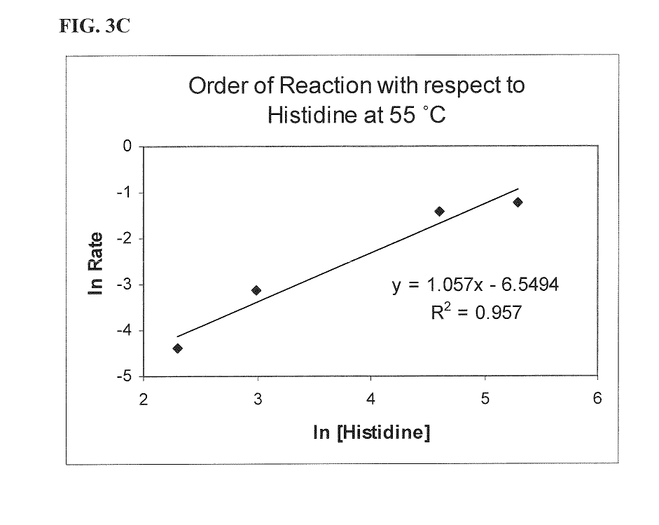

The rate of color change over time was monitored and the initial, linear rates at each temperature were used in Arrhenius analysis to estimate the activation energy (Ea) of the reaction. For example, as shown in FIG. 3A, the estimated Ea from the Arrhenius analysis of 200 mM histidine, pH 7, was determined to be 73.3 kJ/mol. The estimated Ea for 20 mM and 100 mM histidine was determined to be 72.6 and 82.6, respectively (Table 3). The Ea was then used for each concentration of histidine to create a temperature predictive model of the observed color change (see Table 3). Therefore, because histidine buffer yellowing fits Arrhenius modeling, the color change of a refrigerated sample can be predicted from high temperature experiments.

TABLE 3
Results of kinetic evaluation of factors effecting histidine
buffer yellowing.

| | | Time to Reach b* = X | | | | |
|---|---|---|---|---|---|---|
| [Histidine] | Ea | Temp | | Predicted | Actual | |
| (mM) | (kJ/mol) | (° C.) | b* = X | (days) | (days | % Difference |
| 20 | 72.6 | 25 | 1.3 | 428 | 502 | −14.7% |
| | | 40 | 1.6 | 130 | 187 | −30.5% |
| | | 55 | 4.2 | 93 | 117 | −20.5% |
| 100 | 82.6 | 25 | 5.8 | 397 | 502 | −20.9% |
| | | 40 | 6.3 | 88 | 84 | 4.8% |
| | | 55 | 8.1 | 27 | 41 | −34.1% |
| 200 | 73.3 | 5 | 0.9 | 346 | 502 | −31.1% |
| | | 25 | 3.9 | 177 | 187 | −5.3% |
| | | 40 | 6.8 | 75 | 84 | −10.7% |
| | | 55 | 15.4 | 47 | 56 | −16.1% |

The rates of color change of formulations containing varying histidine concentrations were also compared to determine the order of reaction. As shown in FIG. 3B, histidine buffer yellowing is approximately first order at 25° C. (Yellowing rate=k [Histidine]^{l}). However, the curvature at 55° C. suggests a more complex reaction order (FIG. 3C). Similar experiments can be performed to determine the order of reaction with regard to various excipients, e.g., without limitation, polysorbate 80, glycine, glycerol, and/or oxygen content.

### Example 5

Evaluation of Yellow Color Formation as a Result of an Oxidative Mechanism

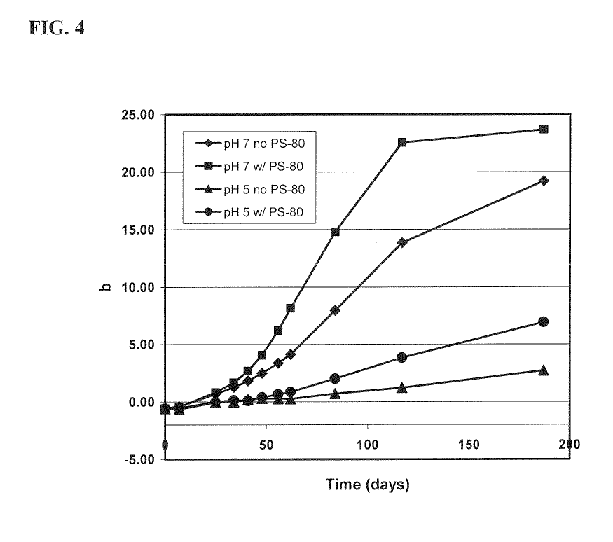

Because polysorbate 80 is a source of peroxides, the effect of oxidation on the yellow color formation of histidine buffer was evaluated. The rate of yellow color formation of 200 mM histidine buffer at 40° C. was monitored at pH 5.0 and at pH 7.0, either in the presence or absence of polysorbate 80. As shown in FIG. 4, polysorbate 80 and the higher pH of 7.0 increased solution yellowing both independently (compare circles for polysorbate 80 with diamonds for pH 7.0), and in combination (squares), compared to histidine buffer at pH 5.0 (triangles).

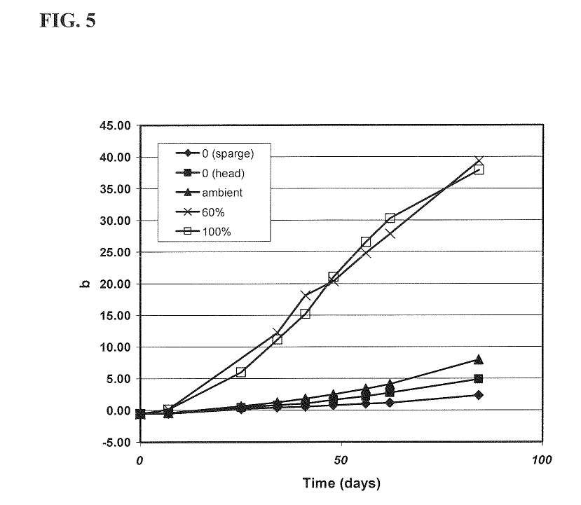

To further analyze the effect of oxidation on yellow color formation, the oxygen content of the histidine buffer and the container headspace was manipulated. The rate of yellow color formation of 200 mM histidine buffer at 40° C. was monitored with some of the formulations overlaid or sparged with 100% nitrogen gas or overlaid with 60% oxygen gas or 100% oxygen gas from Airgas. As shown in FIG. 5, the presence of headspace oxygen was found to significantly increase solution yellowing. Solutions that were capped with ambient headspace (filled triangles), sparged with N₂ (filled diamonds), or overlaid with N₂ (filled squares) showed much less yellow color formation than those that were overlaid with 60% O₂ (crosses) or 100% O₂ (clear squares). Thus, although not intending to be bound by any particular theory, the effect of pH, polysorbate 80, and headspace gas content suggest an oxidative pathway for histidine yellowing.

### Example 6

### Use of Methionine Retards Histidine Buffer Yellowing

In the DOE parameter screen, methionine was shown to retard yellowing of histidine buffer (see Table 2). To determine if methionine could similarly retard the yellowing of histidine buffer containing common protein formulation excipients, including polysorbate 80 and glycine, in various container closure systems, a comparison screen was set up as shown in Table 4.

TABLE 4
Formulation parameters for comparison screen.

| Formulation | 0.05% TWEEN ™ | 0.05% TWEEN ™ | 10 mM |
|---|---|---|---|
| # | 80 (NOF) | 80 (J. T. Baker) | Methionine |
| 1 | | | |
| 2 | | | X |
| 3 | X | | |
| 4 | X | | X |
| 5 | | X | |
| 6 | | X | X |

Formulations containing 50 mM histidine (pH 6.0), 100 mM glycine, 0.05% polysorbate 80 (TWEEN® 80) from either NOF America or J. T. Baker, and 10 mM methionine were filled into glass vials; LUER-LOK™, Hypak SCF™ 1 mL syringes (Becton Dickinson); or staked-needle, Hypak SCF™ 1 mL syringes (Becton Dickinson). Syringes and vials were incubated at 40° C./75% RH and protected from light for 6 months. Solution color was analyzed by expelling the solution from the syringe or removing the solution from the vial and transferring to a 1-cm cell-path quartz cuvette for measurement using the HunterLab ColorQuest XE.

As shown in the DOE parameter screen, methionine was able to significantly retard histidine buffer yellowing both in the absence and presence of polysorbate 80 in all container closure systems tested (see Table 5). As similarly shown in Table 2 and FIG. 4, polysorbate 80 increased histidine buffer yellowing (see Table 5). The use of ultra-pure polysorbate 80 from NOF America in the histidine buffer did not significantly effect yellow color formation in comparison to a lower purity polysorbate 80 obtained from J. T. Baker. As discussed above, yellow color formation seems to be, at least in part, a result of an oxidative mechanism. Although not wishing to be bound by any particular theory, methionine, which is an antioxidant, appears to retard histidine buffer yellowing presumably by acting as an oxygen scavenger.

TABLE 5
Histidine buffer yellowing (b* Values).

| Formulation | LUER-LOK ™ | Staked | Vial |
|---|---|---|---|
| Histidine & Glycine | 0.825 | 1.24 | 1.225 |
| + Methionine | 0.42 | 0.59 | 0.47 |
| % Difference | 49% | 52% | 62% |
| +NOF TWEEN ™ 80 | 3.33 | 5.15 | 7.29 |
| +NOF TWEEN ™ 80 & Methionine | 0.425 | 2.805 | 2.41 |
| % Difference | 87% | 46% | 67% |
| +J. T. Baker TWEEN ™ 80 | 3.18 | 4.765 | 5.69 |
| +J. T. Baker TWEEN ™ 80 & | 0.63 | 3.53 | 2.49 |
| Methionine | | | |
| % Difference | 80% | 26% | 56% |

Table 5 shows results for LUER-LOK™, Hypak SCF™ 1 mL syringes (“LUER-LOK™'”); staked-needle, Hypak SCF™ 1 mL syringes (“Staked”); and glass vials (“Vial”) that contain 50 mM histidine (pH 6.0) and 100 mM glycine (“Histidine & Glycine”) and which were incubated at 40° C./75% RH and protected from light for 6 months. Syringes and vials additionally containing 10 mM methionine (“+Methionine”) were also evaluated. Syringes and vials containing 0.05% polysorbate 80 from NOF America (“+NOF TWEEN™ 80”) as well as syringes and vials marked containing 0.05% polysorbate 80 from NOF America and 10 mM methionine (“+NOF TWEEN™ 80 & Methionine”) were evaluated. Syringes and vials containing 0.05% polysorbate 80 from J. T. Baker (“+J. T. Baker TWEEN™ 80”) as well as syringes and vials containing 0.05% polysorbate 80 from J. T. Baker with 10 mM methionine (“+J. T. Baker TWEEN™ 80 & Methionine”) were also evaluated.

### Example 7

### Correlation of Yellowing with Peroxide Formation, Head Space Volume, Head Space Gas, and Solution Content

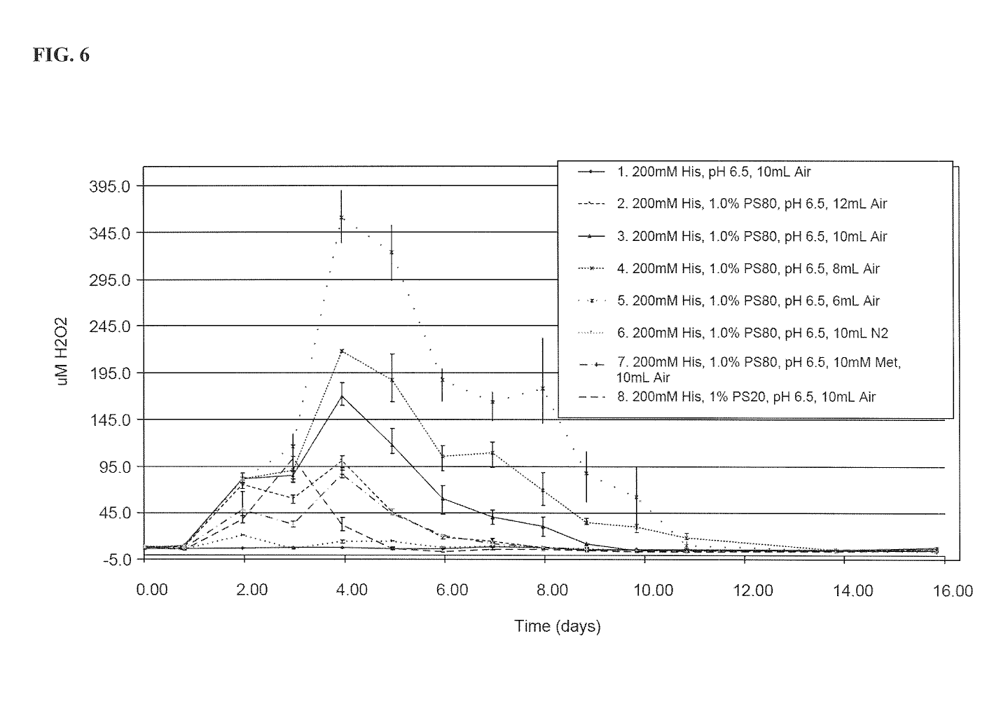

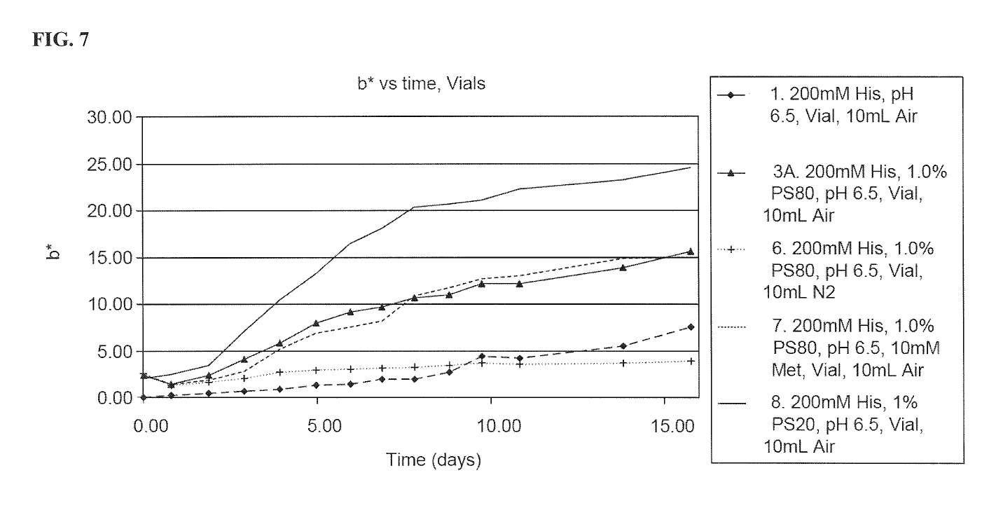

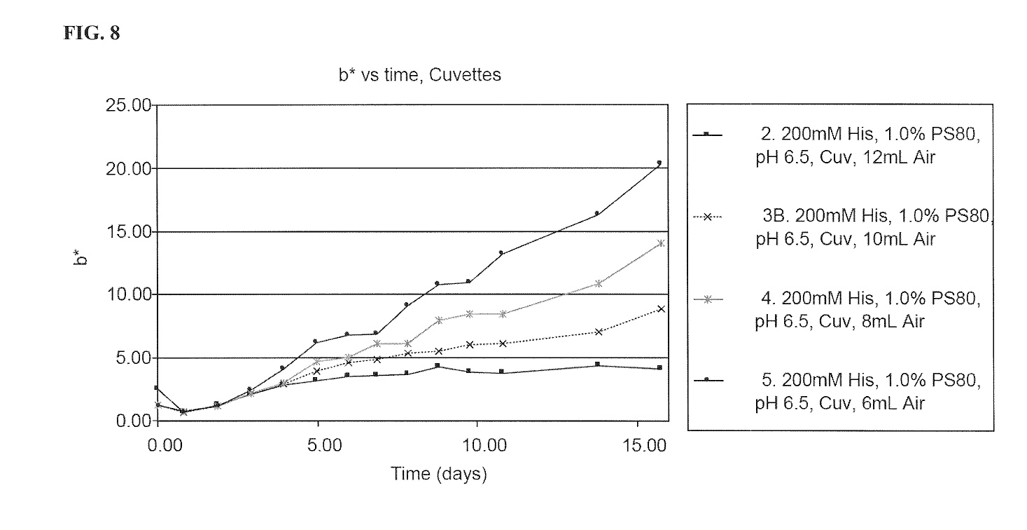

Solutions of 200 mM histidine, 0.05% (w/v) polysorbate 80, pH 5 with varying headspace gas content were incubated in 10 mL glass vials at 60° C. Solution color was measured using the Hunter LAB ColorQuest XE instrument (either in 10 mL vials or in cuvettes if sufficient volume was not available) (see, FIGS. 7-10) and the solution peroxide content was determined using a PEROXOQUANT™ Kit (Thermo Scientific, Waltham, Mass., USA). (See, FIG. 6).

Solution yellowing generally followed a bi-phasic formation where the initial rate of yellowing is faster than the later rate. Concurrently, the peroxide level in the solution first increases and then decreases over time. The observed change in yellowing rate roughly correlates in time to the decrease in peroxide content, suggesting that peroxide may, at least partly, be driving the color change.

Headspace gas content also contributed to both solution yellowing and peroxide content. See, FIGS. 6-10. Vials were filled with varying volumes of solution (6, 8, 10 or 12 mL) with air in the headspace. Samples containing less liquid (and therefore having greater headspace) yellowed more appreciably and contained more peroxide. One set of samples were formulated with N2 in the headspace rather than air. These solutions did not yellow significantly and also had negligible peroxide content.

Polysorbate significantly increased solution yellowing and it was shown that solutions that did not contain polysorbate also had negligible peroxide content. See, FIGS. 6-8. These results suggest that the acceleration in solution yellowing by polysorbate 80 may correlate to peroxide impurities that polysorbates are known to contain.

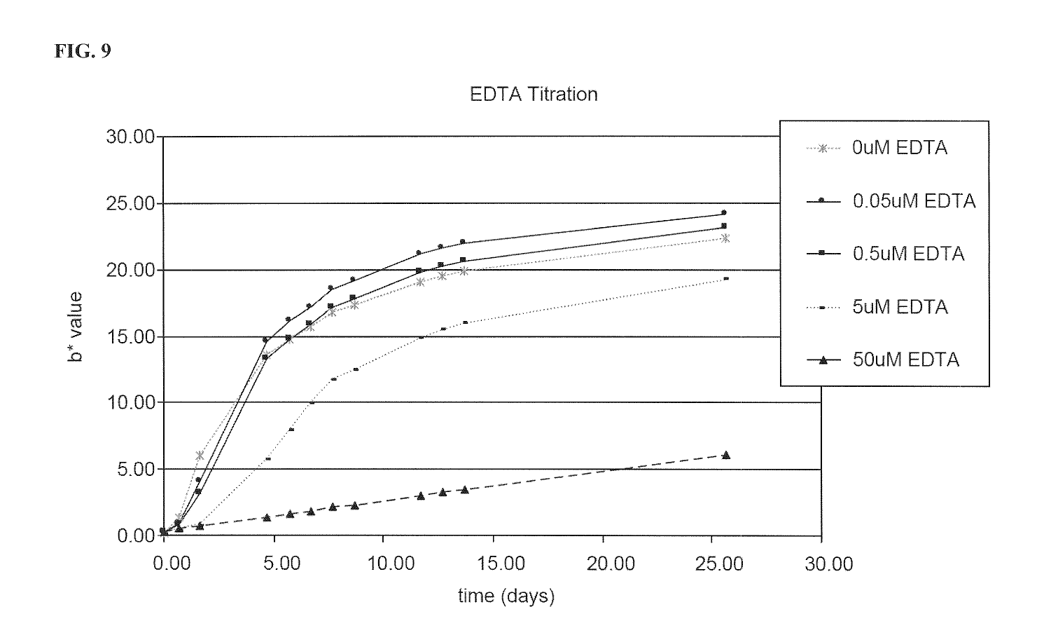

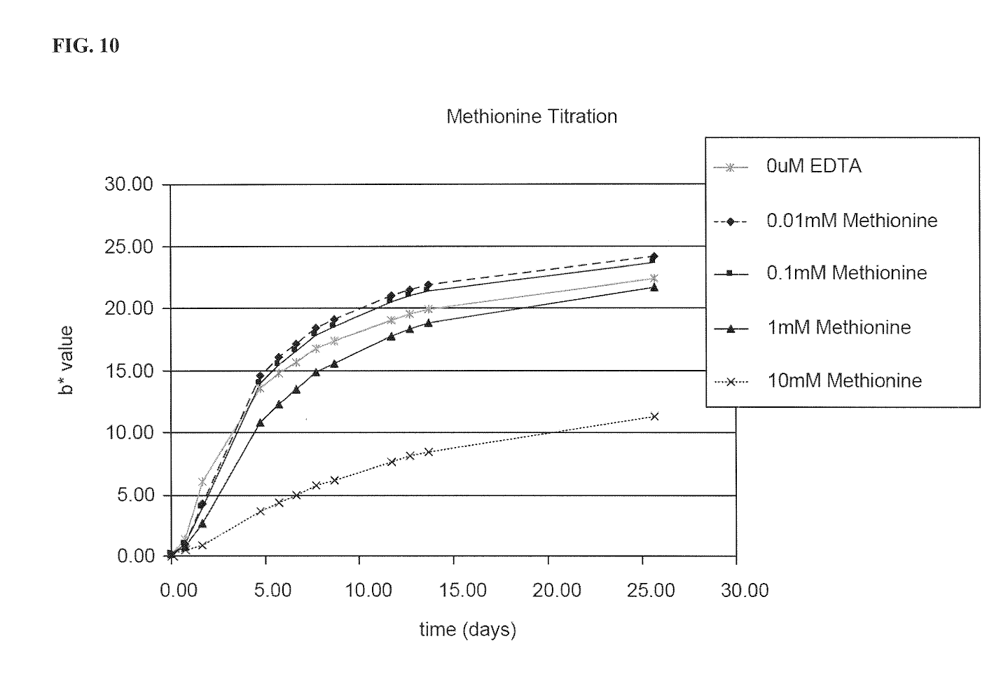

In contrast, EDTA effectively retarded solution yellowing. As little as 50 μM EDTA was able to significantly retard solution yellowing. See, FIG. 9. Likewise, methionine also effectively retarded solution yellowing. As little as 10 μM methionine was able to significantly retard solution yellowing. See, FIG. 10.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method for preventing or retarding yellow color or peroxide formation in a composition, wherein the method comprises use of an antioxidant or oxygen scavenger in said composition, wherein said composition comprises a solution or formulation selected from the group consisting of:
a) a buffer solution;
b) a protein formulation;
c) a solution containing a protein; and
d) a solution containing an antibody.

2. A method of reducing or decreasing the amount of yellow color or peroxide in a composition, wherein the method comprises use of an antioxidant or oxygen scavenger in said composition, wherein said composition comprises a solution or formulation selected from the group consisting of:
a) a buffer solution;
b) a protein formulation;
c) a solution containing a protein; and
d) a solution containing an antibody.

3. The method of claim 1, wherein said composition comprises a compound selected from the group consisting of:
a) histidine;
b) citrate;
c) phosphate;
d) succinate;
e) acetate;
f) Tris; and
g) any combination of two or more of a, b, c, d, e, and f.

4. (canceled)

5. The method of claim 1, wherein said composition comprises an excipient, wherein said excipient is selected from the group consisting of:
a) a polysorbate compound;
b) polysorbate 20;
c) polysorbate 80;
d) NaCl;
e) sucrose;
f) glycerol;
g) arginine;
h) glycine;
i) trehalose;
j) mannitol;
k) xylitol;
l) lactose;
m) sorbitol;
n) a poloxamer;
o) a glycol;
p) CaCl₂;
q) imidazole;
r) benzyl alcohol;
s) urea;
t) leucine;
u) isoleucine;
v) threonine;
w) glutamate or glutamic acid;
x) phenylalanine;
y) cresol; and
z) any combination of two or more of a through y.

6. The method of claim 1, wherein said antioxidant or oxygen scavenger is selected from the group consisting of:
a) methionine;
b) ascorbic acid;
c) glutathione;
d) Vitamin A;
e) Vitamin E;
f) selenium;
g) retinyl palmitate;
h) cysteine;
i) sodium sulfite;
j) thioglycerol;
k) thioglycolic acid;
l) metabisulfite; and
m) any combination of two or more of a through l.

7. (canceled)

8. The method of claim 1, wherein the concentration of said antioxidant or oxygen scavenger is selected from the group consisting of:
a) about 0.0001 mM to about 10000 mM;
b) about 0.001 mM to about 1000 mM;
c) about 0.01 mM to about 100 mM; and
d) about 0.1 mM to about 10 mM.

9. The method of claim 1, wherein said composition further comprises a chelating agent.

10. The method of claim 9, wherein said chelating agent is selected from the group consisting of:
a) EDTA (ethylenediaminetetraacetic acid);
b) EGTA (ethyleneglycoltetraacetic acid);
c) ascorbic acid;
d) iminodiacetate;
e) tetrasodium iminodisuccinate;
f) citric acid;
g) dicarboxymethylglutamic acid;
h) EDDS (ethylenediaminedisuccinic acid);
i) DTPMP.Na (hepta sodium salt of diethylene triamine penta or methylene phosphonic acid);
j) malic acid;
k) NTA (nitrilotriacetic acid);
l) a nonpolar amino acid;
m) methioninie;
n) oxalic acid;
o) phosphoric acid;
p) a polar amino acid;
q) arginine;
r) asparagine;
s) aspartic acid;
t) glutamic acid;
u) glutamine;
v) lysine;
w) ornithine;
x) a siderophore;
y) Desferrioxamine B;
z) succinic acid;
aa) hydrolysed wool;
bb) a chelating resin; and
cc) a CHELEX® resin.

11. The method of claim 1, further comprising decreasing exposure of said composition to oxygen, wherein said decreasing exposure is performed by a means selected from the group consisting of:
a) reducing the headspace gas content between the surface of the composition and a container closure;
b) reducing ambient oxygen content;
a) overlaying the composition with nitrogen;
d) sparging the composition with nitrogen, and
e) any combination of a, b, c, or d.

12. (canceled)

13. (canceled)

14. The method of claim 1, further comprising adjusting the pH of said composition, wherein the adjusted pH is other than about pH 6.0.

15. The method of claim 1, further comprising adjusting the pH of said composition, wherein the adjusted pH is selected from the group consisting of:
a) about 7.5 to about 7.0;
b) about 7.0 to about 6.5;
c) about 6.0 or less;
d) about 5.5 or less; and
e) about 5.0 or less.

16. The method of claim 1, further comprising use of container coloration and/or packaging to protect said composition from light.

17. (canceled)

18. A method for preventing or retarding yellow color or peroxide formation in a composition, the method comprising use of at least two factors to prevent or retard yellow color or peroxide formation, wherein said composition is selected from the group consisting of:
a) a buffer solution;
b) a protein formulation;
c) a solution containing a protein; and
d) a solution containing an antibody.

19. A method of reducing or decreasing the amount of yellow color or peroxide in a composition, wherein the method comprises use of at least two factors to reduce or decrease yellow color or peroxide formation, wherein said composition is selected from the group consisting of:
a) a buffer solution;
b) a protein formulation;
c) a solution containing a protein; and
d) a solution containing an antibody.

20. The method of claim 18, wherein said use of at least two factors comprises use of any combination of two or more factors selected from the group consisting of:
a) methionine;
b) decreased oxygen exposure;
c) NaCl;
d) a polysorbate compound;
e) polysorbate 20;
f) polysorbate 80;
g) arginine;
h) a pH of about 5.0;
i) a pH of about 5.5;
j) a pH of about 6.0;
k) a pH of about 6.5; and
l) a pH of about 7.0.

21. The method of either claim 18, wherein said use of at least two factors comprises use of one or more combinations selected from the group consisting of:
a) methionine and decreased oxygen exposure;
b) methionine and a pH of about 5;
c) methionine and a pH of about 6;
d) NaCl and polysorbate 80; and
e) arginine and a pH of about 7.

22. The method of claim 2, wherein said reduction or decrease in the amount of yellow color in a composition is measured as a percent decrease in b* value.

23. (canceled)

24. A method for predicting or determining the rate of yellow color or peroxide formation in a composition, wherein said composition comprises a solution or formulation selected from the group consisting of:
a) a buffer solution;
b) a protein formulation;
c) a solution containing a protein; and
d) a solution containing an antibody
and wherein the predicting or determining comprises the steps of incubating the composition at a specified range of temperatures, quantitating the amount of yellow color or peroxide formed as a function of time and temperature, and extrapolating a prediction or determination of the rate of yellow color or peroxide formation in said composition for any temperature inside or outside the specified range of temperatures.

25. The method of claim 24, wherein the method further comprises the steps of preparing a range of concentrations of the composition and extrapolating a prediction or determination of the rate of yellow color or peroxide formation in said composition for any concentration inside or outside the specified range of concentrations.

26. The method of claim 24, wherein said composition comprises a compound selected from the group consisting:
a) histidine;
b) citrate;
c) phosphate;
d) succinate;
e) Tris;
f) acetate; and
g) any combination of two or more of a, b, c, d, e, and f.

27. The method of claim 24, wherein the rate of yellow color or peroxide formation in said composition is predicted or determined as a function of composition storage temperature.

28. (canceled)

29. The method of claim 24, wherein the prediction or determination is calculated based on a two-factor interaction.

30. (canceled)
